(19) United States
(12) Patent Application Publication
(10) Pub. No.: US 2007/0014866 A1
(43) Pub. Date: Jan. 18, 2007

(54) Composition for improving the efficacy and reducing the side effects of omega 3 fatty acids, fish oils and cardiovascular and diabetic treatments

(76) Inventor: Curt Hendrix, Encino, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/484,230

(22) Filed: Jul. 10, 2006

Related U.S. Application Data
(60) Provisional application No. 60/699,669, filed on Jul. 15, 2005.

(51) Int. Cl. A61K 35/60; A61K 31/7034; A61K 31/675; A61K 31/355; A61K 31/366; A61K 31/22; A61K 31/205; A61K 31/195; A61K 31/12; A61K 31/401

(52) U.S. Cl. 424/523; 514/547; 514/89; 514/25; 514/423; 514/460; 514/458; 514/554; 514/561; 514/690; 514/682

(57) ABSTRACT

Synergistic therapeutic compositions for reducing triglycerides, lowering LDL and increasing HDL are formed by combining either pantethine or CoA, or a combination of pantethine and CoA with fish oils. Either pantethine or CoA, or a combination of pantethine and CoA, added to cardiovascular drugs or compositions for lowering cholesterol increases the therapeutic effects and decreasing the side effects of those drugs or compositions. Either pantethine or CoA, or a combination of pantethine and CoA, added to drugs or compositions used in the treatment of Type I or Type II diabetes also increases the therapeutic effects and decreasing the side effects of those drugs or compositions.

This application claims the benefit of U.S. Provisional Application No. 60/699,669 filed Jul. 15, 2005.

Research has shown that daily dosages of omega 3 polyunsaturated fatty acids, which include but are not limited to Alpha linolenic acid (ALA) 18:3 N=3, Eicosapentanoic acid (EPA) 20:5 N=3 and Docosahexanoic Acid (DHA) 22:6 N=3 have significant anti-inflammatory and cardiovascular health benefits in humans. In regard to these compounds, the 18, 20: and 22 designate the number of carbons in the fatty acid chain. The second number (3, 5, and 6 respectively designates the number of double bonded carbons in the chain, and the N number is designates the carbon atom where the first double bond start, counting from the non-carboxylic end of the acid. Fish oils usually contain combinations of EPA and DHA in varying ratios.

Studies have shown that very high doses of fish oils containing EPA and DHA, in the range of 2 or more grams, can be used to reduce triglycerides. A shortcoming of this approach is that only 25-45% of fish oils constitute EPA and DHA. Studies have further shown that at least 3000 mg of EPA and/or DHA are needed to generate significant reductions in triglycerides. At a 30% concentration for these compounds in fish oil, 9 grams or 10-15 large softgel capsules would have to be taken a day to achieve this dosing. As a result, patient compliance would be unacceptable because most people will not stick with a medication or dietary supplement that requires 10-15 large capsules to be swallowed each day of their lives.

Secondly, studies have shown that many people who do in fact take the fish oils demonstrate an increase in LDL cholesterol which has negative consequences for heart disease. Further, studies show that fish oils have no significant impact on raising HDL cholesterol, which would be a desirable result.

Pantothenic acid (PA), a B-complex vitamin, is essential for humans and animals for growth, reproduction, and normal physiological functions. It is a precursor of the coenzymes, CoA and acyl carrier protein of fatty acid synthase, which are involved in more than 100 different metabolic pathways including energy metabolism of carbohydrates, proteins and lipids, and the synthesis of lipids, neurotransmitters, steroid hormones, porphyrins and hemoglobin.

Pantethine is not a vitamin. However it is a derivative of pantothenic acid. Pantethine comprises two molecules of pantetheine joined by a disulfide bond (chemical bond between two molecules of sulfur). In the synthetic pathway for the formation of coenzyme A (CoA), pantethine is closer to CoA than pantothenic acid, and is the functional component of CoA and acyl carrier proteins. Pantethine has been reported by a number of investigators to have a cholesterol lowering effect. Several studies have found that 900 mg dosages of pantethine delivered on a daily basis, preferably 300 mg, three times daily, to be significantly more effective than placebo in lowering total cholesterol and trigylceride levels in the blood of both diabetic and non-diabetic individuals. Pantethine was also found to lower cholesterol and triglyceride levels in diabetic patients on hemodialysis without adverse side effects. Because of the low incidence of side effect of pantethine and the increased risk of drug toxicity in patients with renal (kidney) failure, pantethine is especially attractive for delivery to hemodialysis patients.

U.S. Pat. No. 6,509,035 discusses the problems in orally delivering beneficial amounts of CoA because of dephosphorylation in the enteric tract and discloses oral deliver of a combination of up to 10% CoA with an oxidant and an acid buffer or acidifier to address this problem.

Applicant has discovered that the addition of pantethine and/or CoA to many compounds, pharmaceuticals and nutritional products, particularly when used to prevent, reverse or treat cardiovascular diseases and diabetic conditions (i.e., delivery at the same time or in a single dosage) can synergistically lower the dosage of those compounds, pharmaceuticals or nutritional products necessary to obtain the same beneficial effect and, at the same time, reduce LDL and total cholesterol and increase HDL in the patient.

For example, combining pantethine and/or CoA with fish oils synergistically lowers the dosage of both fish oils and pantethine or CoA necessary to achieve significant reductions in triglycerides. Because a lower dosage or fewer capsules on a daily basis is required, patient compliance significantly increases. Additionally, adding pantethine and/or CoA to fish oils not only counteracts the undesirable, elevated LDL levels seen with fish oils; it results in lower LDL. Still further, while the delivery of fish oils alone has no effect on HDL levels, the combination results in a significant increased in HDL, which is a highly desired effect.

A recommended oral daily dosage of the pantithine and/or CoA in combination with fish oil comprises from about 100 mg to about 1200 mg of pantethine from about 1 mg to about 1000 mg of CoA and from about 1 gr to about 10 gr of fish oil, one-third of said dosage delivered three times a day. A more preferred oral daily dosage would comprise a total of from about 600 mg to about 900 mg of pantethine from about 1 mg to about 500 mg of CoA and from about 1 gr to about 7 gr of fish oil.

However, as indicated above, the beneficial, synergistic effect of adding pantethine and/or CoA is not limited to fish oil. Beneficial effects can be obtained by adding pantethine and/or CoA to cardiovascular and cholesterol lowering drugs such as statins, fibrates, bile acid sequesterants, niacin, acarbose, cholesteryl ester transfer protein inhibitors, PPAR and LXR receptor agonists, thiol-containing compounds, tocotrienols, glucaric acid and its salts, taurine, sesamin lignin, beta-ionone, pyridoxal-5-phosphate, vitamin K and carnosic acid.

Additional synergisym occurs when pantethine and/or CoA is added to drugs and compounds for Type I and Type II diabetes such as: insulin, biguanides, sulphonylurea, thiazolidinediones, meglitinides, chromium, vanadium compounds, thiamin, other Vitamin B-1 derivatives and forms such as thiamine pyrophosphate, benfotiamine, pyridoxamine, anti-glycation agents, such as alpha-lipoic-acid, 1-carnosine, vitamin D, and biotin.

1. A therapeutic composition for reducing triglycerides, lowering LDL and increasing HDL comprising a combination of either pantethine or CoA, or pantethine and CoA with fish oils.

2. The therapeutic composition of claim 1 wherein the fish oils contain EPA and DHA.

3. The therapeutic composition of claim 1 wherein a twenty four hour dosage comprises from about 100 mg to about 1200 mg of pantethine, from about 1 mg to about 1000 mg of CoA and from about 1 gr to about 10 gr of fish oil.

4. The therapeutic composition of claim 3 wherein the twenty four hour dosage is delivered as three smaller dosages, each smaller dosage comprising one-third of the 24 hour dosage.

5. The therapeutic composition of claim 1 wherein a twenty four hour dosage comprises from about 600 mg to about 900 mg of pantethine, from about 1 mg to about 500 mg of CoA and from about 1 gr to about 7 gr of fish oil.

6. A synergistic composition for increasing the therapeutic effects and decreasing the side effects of cholesterol lowering drugs comprising pantethine or CoA, or a combination of pantethine and CoA in combination with cholesterol lowering drugs or compounds.

7. The synergistic combination of claim 6 wherein the cholesterol lowering drugs or compounds are chosen from the group consisting of statins, fibrates, bile acid sequesterants, niacin, acarbose, cholesteryl ester transfer protein inhibitors, PPAR and LXR receptor agonists, thiol-containing compounds, tocotrienols, glucaric acid and its salts, taurine, sesamin lignin, beta-ionone, pyridoxal-5-phosphate, vitamin K, carnosic acid and fish oils.

8. A synergistic combination for increasing the therapeutic effects and decreasing the side effects of drugs and compounds used for treating Type I and Type II diabetes comprising pantethine or CoA, or a combination of pantethine and CoA combined with said drugs and compounds.

9. The synergistic combination of claim 8 wherein drugs and compounds used for treating Type I and Type II diabetes are selected from the group consisting of insulin, biguanides, sulphonylurea, thiazolidinediones, meglitinides, chromium, vanadium compounds, thiamin, vitamin B-1 derivatives and anti-glycation agents.

10. The synergistic combination of claim 9 wherein the vitamin B-1 derivatives comprise thiamine pyrophosphate, benfotiamine or pyridoxamine,

11. The synergistic combination of claim 9 wherein the anti-glycation agents comprise alpha-lipoic-acid, 1-carnosine, vitamin D or biotin.
